SEQUENCE LISTING (51 sequences)

SEQ ID NO: 1 - DNA, 33 bp, Artificial
  Synthetic polynucleotide sequence
catctcttctccgagccggtcgaaatagtgagt

SEQ ID NO: 2 - DNA, 20 bp, Artificial
  Synthetic polynucleotide sequence
actcactataggaagagatg

SEQ ID NO: 3 - DNA, 44 bp, Artificial
  Synthetic polynucleotide sequence
actcatctgtgaactcactataggaagagatgtgtcaactcgtg

SEQ ID NO: 4 - DNA, 12 bp, Artificial
  Synthetic polynucleotide sequence
cacgagttgaca

SEQ ID NO: 5 - DNA, 12 bp, Artificial
  Synthetic polynucleotide sequence
gtgctcaactgt

SEQ ID NO: 6 - DNA, 22 bp, Artificial
  Synthetic polynucleotide sequence
cacgagttgacacatctcttcc

SEQ ID NO: 7 - DNA, 22 bp, Artificial
  Synthetic polynucleotide sequence
tatagtgagttcacagatgagt

SEQ ID NO: 8 - DNA, 20 bp, Artificial
  Synthetic polynucleotide sequence
cgagttgacacatctcttcc

SEQ ID NO: 9 - DNA, 20 bp, Artificial
  Synthetic polynucleotide sequence
tatagtgagttcacagatga

SEQ ID NO: 10 - DNA, 18 bp, Artificial
  Synthetic polynucleotide sequence
agttgacacatctcttcc

SEQ ID NO: 11 - DNA, 18 bp, Artificial
  Synthetic polynucleotide sequence
tatagtgagttcacagat

SEQ ID NO: 12 - DNA, 16 bp, Artificial
  Synthetic polynucleotide sequence
ttgacacatctcttcc

SEQ ID NO: 13 - DNA, 16 bp, Artificial
  Synthetic polynucleotide sequence
tatagtgagttcacag

SEQ ID NO: 14 - DNA, 14 bp, Artificial
  Synthetic polynucleotide sequence
gacacatctcttcc

SEQ ID NO: 15 - DNA, 14 bp, Artificial
  Synthetic polynucleotide sequence
tatagtgagttcac

SEQ ID NO: 16 - DNA, 23 bp, Artificial
  Synthetic polynucleotide sequence
cacgagttgacacatctcttcct

SEQ ID NO: 17 - DNA, 21 bp, Artificial
  Synthetic polynucleotide sequence
atagtgagttcacagatgagt

SEQ ID NO: 18 - DNA, 21 bp, Artificial
  Synthetic polynucleotide sequence
cgagttgacacatctcttcct

SEQ ID NO: 19 - DNA, 19 bp, Artificial
  Synthetic polynucleotide sequence
atagtgagttcacagatga

SEQ ID NO: 20 - DNA, 19 bp, Artificial
  Synthetic polynucleotide sequence
agttgacacatctcttcct

SEQ ID NO: 21 - DNA, 17 bp, Artificial
  Synthetic polynucleotide sequence
atagtgagttcacagat

SEQ ID NO: 22 - DNA, 17 bp, Artificial
  Synthetic polynucleotide sequence
ttgacacatctcttcct

SEQ ID NO: 23 - DNA, 15 bp, Artificial
  Synthetic polynucleotide sequence
atagtgagttcacag

SEQ ID NO: 24 - DNA, 15 bp, Artificial
  Synthetic polynucleotide sequence
gacacatctcttcct

SEQ ID NO: 25 - DNA, 13 bp, Artificial
  Synthetic polynucleotide sequence
atagtgagttcac

SEQ ID NO: 26 - DNA, 15 bp, Artificial
  Synthetic polynucleotide sequence
nnnnnnnngnnnnnn

SEQ ID NO: 27 - DNA, 15 bp, Artificial
  Synthetic polynucleotide sequence
yyyyaatacgnnnnn

SEQ ID NO: 28 - DNA, 15 bp, Artificial
  Synthetic polynucleotide sequence
nnnnnnaugnnnnnn

SEQ ID NO: 29 - DNA, 15 bp, Artificial
  Synthetic polynucleotide sequence
nnnnnnnrynnnnnn

SEQ ID NO: 30 - DNA, 120 bp, Artificial
  Synthetic polynucleotide sequence
gatgtgtccgtgcaggttcgaggaagagatggcgacgtggaacccatgatgagccgagttggggtgtgtctctcgtatatggcggaagtgggacaatagttgagtagctgatcctgatgg

SEQ ID NO: 31 - DNA, 121 bp, Artificial
  Synthetic polynucleotide sequence
gatgtgtccgtgcaggttcgaggaagagatggcgacatcggacaagggaggcactggaggttgaggtagtgagcgttggttaacgccggacaaagggaagcatggtagctgatcctgatgg

SEQ ID NO: 32 - DNA, 58 bp, Artificial
  Synthetic polynucleotide sequence
aagaatcgttgtcattggcacacggaggtttactgagtggtaaccacgttgcatggaa

SEQ ID NO: 33 - RNA, 97 nt, Artificial
  Synthetic polynucleotide sequence
ggauaauagccguagguugcgaaagcgacccugaugagaagccaaagccguagcgcagaugaucucgccaucaguaccgaaacgguagcgagagcuc

SEQ ID NO: 34 - DNA, 105 bp, Artificial
  Synthetic polynucleotide sequence
ctgcagaattctaatacgactcactataggaagagatggcgacatctctggttccctgttggtagggttatcgttcggatcttagtgtgtcggtaagcttggcac

SEQ ID NO: 35 - DNA, 107 bp, Artificial
  Synthetic polynucleotide sequence
gggacgaattctaatacgactcactataggaagagatggcgagatctcttgtattagctacactgttagtgcatcgtttttaatctcgtggacggtaagcttggcac

SEQ ID NO: 36 - RNA, 14 nt, Artificial
  Synthetic polynucleotide sequence
gccguagguugccc

SEQ ID NO: 37 - RNA, 13 nt, Artificial
  Synthetic polynucleotide sequence
acgagucaggauu

SEQ ID NO: 38 - RNA, 90 nt, Artificial
  Synthetic polynucleotide sequence
ggauaauagccguagguugcgaaagcgacccugaugagcccugcgaugcagaaaggugcugacgacacaucgaaacgguagcgagagcuc

SEQ ID NO: 39 - RNA, 90 nt, Artificial
  Synthetic polynucleotide sequence
ggauaauagccguagguugcgaaagcgacccugaugagccuuuagggccaaguguggugaaagacacacucgaaacgguagcgagagcuc

SEQ ID NO: 40 - RNA, 90 nt, Artificial
  Synthetic polynucleotide sequence
ggauaauagccguagguugcgaaagcgacccugaugagccuguggaaacagacguggcacaugacuacgucgaaacgguagcgagagcuc

SEQ ID NO: 41 - RNA, 88 nt, Artificial
  Synthetic polynucleotide sequence
ggauaauagccguagguugcgaaagcgacccugaugagccuuaggauaugcaugaugcagaaggacgucgaaacgguagcgagagcuc

SEQ ID NO: 42 - RNA, 92 nt, Artificial
  Synthetic polynucleotide sequence
ggauaauagccguagguugcgaaagcgacccugaugaucuggauaccaugcaugaugcaccuuggcagucuuagaaacgguagcgagagcuc

SEQ ID NO: 43 - RNA, 99 nt, Artificial
  Synthetic polynucleotide sequence
ggauguccagucgcuugcaaugcccuuuuagacccugaugagcaggcaaacgugcgccuagaaugcagacaccaacgaaacggugaaagccguaggucu

SEQ ID NO: 44 - RNA, 99 nt, Artificial
  Synthetic polynucleotide sequence
ggauguccagucgcuugcaaugcccuuuuagacccugaugaggaucaucggacuuuguccuguggaguaagaucgcgaaacggugaaagccguaggucu

SEQ ID NO: 45 - DNA, 28 bp, Artificial
  Synthetic polynucleotide sequence
nnnnnntccgagccggtcgaannnnnnn

SEQ ID NO: 46 - DNA, 22 bp, Artificial
  Synthetic polynucleotide sequence
nnnnnctgggccyyyyrrrrac

SEQ ID NO: 47 - DNA, 24 bp, Artificial
  Synthetic polynucleotide sequence
nnnnnngutgaccccuugnnnnnn

SEQ ID NO: 48 - DNA, 29 bp, Artificial
  Synthetic polynucleotide sequence
nnnnnnrggctagctacaacgannnnnnn

SEQ ID NO: 49 - DNA, 32 bp, Artificial
  Synthetic polynucleotide sequence
gatgtgtccgtgcaggttcgattcttgtgact

SEQ ID NO: 50 - RNA, 65 nt, Artificial
  Synthetic polynucleotide sequence
gggcgacccugaugagugugugggaagaaacuguggcacuucggugccagcgugugcgaaacggu

SEQ ID NO: 51 - RNA, 54 nt, Artificial
  Synthetic polynucleotide sequence
ggguccucugaugagcuuccguuuucagucgggaaaaacugaagcgaaacucgu

(19) United States
(12) Patent Application Publication
(10) Pub. No.: US 2006/0094026 A1
(43) Pub. Date: May 4, 2006

(54) Nucleic acid enzyme light-up sensor utilizing invasive DNA

(76) Inventors: Yi Lu, Champaign, IL (US); Juewen Liu, Urbana, IL (US)

Correspondence Address: EVAN LAW GROUP LLC, 566 WEST ADAMS, SUITE 350, CHICAGO, IL, 60661, US

(21) Appl. No.: 10/980,856

(22) Filed: Nov. 3, 2004

(51) Int. Cl. C12Q 1/68; C12M 1/34

(52) U.S. Cl. 435/6; 435/287.2; 977/924

(57) ABSTRACT

The present invention provides a calorimetric light-up sensor for determining the presence and optionally the concentration of an analyte in a sample. Methods of utilizing the sensor and kits that include the sensor also are provided. The sensor utilizes invasive DNA to assist the analyte dependent disaggregation of an aggregate that includes nucleic acid enzymes, substrates, and particles.

### FEDERALLY SPONSORED RESEARCH OR DEVELOPMENT

This subject matter of this application may have been funded in part under the following research grants and contracts: DOE Grant No. DEFG02-01-ER63179, NSF CTS-0120978, and NSF DMR-0117792. The U.S. Government may have rights in this invention.

### BACKGROUND

The ability to determine the presence of an analyte in a sample is of significant benefit. For example, many metals and metal ions, such as lead, mercury, cadmium, chromium, and arsenic, pose significant health risks when present in drinking water supplies. To prevent the contamination of drinking and other water supplies, it is common to test industrial waste-streams before their release to the water treatment plant. Biological fluids, such as blood and those originating from body tissues, also may be tested for a variety of analytes to determine if the body has been exposed to harmful agents or if a disease state exists. For example, recently there has been the need to detect trace amounts of anthrax and other biologically harmful agents in a variety of samples.

Colorimetric methods are commonly used for the detection of metals and ions in soil, water, waste-streams, biological samples, body fluids, and the like. In relation to instrument based methods of analysis, such as atomic absorption spectroscopy, colorimetric methods tend to be rapid and require little in the way of equipment or user sophistication. For example, colorimetric test are available to aquarists that turn darker shades of pink when added to aqueous samples containing increasing concentrations of the nitrate (NO₃⁻) ion. In this manner, calorimetric tests show that the analyte of interest, such as nitrate, is present in the sample and also may provide an indicator of the amount of analyte in the sample through the specific hue of color generated.

While colorimetric test are extremely useful, they only exist for a limited set of analytes, often cannot detect very small or trace amounts of the analyte, and depending on the nature of the sample, can generate unacceptable levels of false positive or negative results. False positives occur when the colorimetric reagents produce the color associated with the presence of an analyte when the analyte is not present, while false negatives occur when the analyte of interest is present in the sample, but the expected color is not produced. False positives are often the result of constituents in the sample that the colorimetric test cannot distinguish from the analyte of interest. False negatives often result from sample constituents that interfere with the chemical reaction that provides the color associated with the analyte.

As can be seen from the above description, there is an ongoing need for colorimetric tests that can identify trace amounts of a broader scope of analytes. Furthermore, colorimetric tests having a lower incidence of false positive and/or negative results also would provide significant benefit.

### SUMMARY

In one aspect of the invention, a sensor system is disclosed that includes a nucleic acid enzyme, a substrate for the nucleic acid enzyme, first particles, and invasive DNA. The substrate may include first polynucleotides and the first particles may include second polynucleotides that are coupled to the first particles. The invasive DNA may include fourth polynucleotides. The first polynucleotides may be at least partially complementary to the second and fourth polynucleotides. The sensor system also may include second particles that include third polynucleotides that are at least partially complementary to the first polynucleotides.

In another aspect of the invention, a method of detecting an analyte is disclosed that includes combining an aggregate, a sample, and invasive DNA to detect a color change responsive to the analyte. The aggregate may include a substrate and first particles. The aggregate also may include second particles and an endonuclease.

In another aspect of the invention, a kit for detecting an analyte is disclosed that includes a first container containing a system for forming aggregates that includes first polynucleotides and first particles and a second container containing invasive DNA.

In order to provide a clear and consistent understanding of the specification and claims, the following definitions are provided.

The term “sample” or “test sample” is defined as a composition that will be subjected to analysis that is suspected of containing the analyte of interest. Typically, a sample for analysis is in a liquid form, and preferably the sample is an aqueous mixture. A sample may be from any source, such as an industrial sample from a waste-stream or a biological sample, such as blood, urine, or saliva. A sample may be a derivative of an industrial or biological sample, such as an extract, a dilution, a filtrate, or a reconstituted precipitate.

The term “analyte” is defined as one or more substance potentially present in the sample. The analysis process determines the presence, quantity, or concentration of the analyte present in the sample.

The term “colorimetric” is defined as an analysis process where the reagent or reagents constituting the sensor system produce a color change in the presence or absence of an analyte.

The term “sensitivity” refers to the lower concentration limit at which a sensor system can detect an analyte. Thus, the more sensitive a sensor system is to an analyte, the better the system is at detecting lower concentrations of the analyte.

The term “selectivity” refers to the ability of the sensor system to detect the desired analyte in the presence of other species.

The term “hybridization” refers to the ability of a first polynucleotide to form at least one hydrogen bond with at least one second nucleotide under low stringency conditions.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1 represents a colorimetric analytic method of determining the presence and optionally the concentration of an analyte in a sample.

FIG. 2A represents a DNAzyme that depends on Pb(II) as a co-factor to display catalytic activity.

FIG. 2B represents the cleavage of a DNA based substrate by a DNAzyme.

FIG. 3A represents the disaggregation of an aggregate in the presence of a Pb(II) analyte and invasive DNA.

FIG. 3B represents the tail-to-tail hybridization of a DNA based substrate with oligonucleotide functionalized particles.

FIG. 3C represents the head-to-tail hybridization of a DNA based substrate with oligonucleotide functionalized particles.

FIG. 4 is a graph relating extinction ratios to the wavelengths of light emitted from a sample by aggregated (solid line) and disaggregated (dashed line) gold nanoparticles.

FIG. 5A is a graph showing the change in extinction ratios over time for samples containing invasive DNA (Inva) and Pb(II) (∘), invasive DNA (Inva) without Pb(II) (▴), and a control sample containing Pb(II) without invasive DNA (▪).

FIG. 5B is a graph showing the change in extinction ratios over time for samples containing invasive DNA (Inva-A) and Pb(II) (∘), invasive DNA (Inva-A) without Pb(II) (▴), and a control sample containing Pb(II) without invasive DNA (▪).

FIG. 6A is a graph plotting the change in extinction ratios as a function of time for each of the shortened (in relation to the original Inva strand) preferable invasive DNA strands with and without the Pb(II) analyte.

FIG. 6B is a graph plotting the change in extinction ratios as a function of time for each of the shortened (in relation to the original Inva strand) alternate invasive DNA strands with and without the Pb(II) analyte.

FIG. 7A is a graph depicting the ratios of extinction at 522 and 700 nm plotted as a function of time for multiple metal cations.

FIG. 7B is a graph depicting the correlation between the observed extinction ratios for the color change of the sensor system and the concentration of the Pb(II) analyte after five minutes.

FIG. 7C is a graph depicting the extinction ratios for multiple Pb(II) analyte concentrations over a 10 minute time period with Inva-6.

FIG. 8 is a graph depicting the NaCl-dependent stability of gold nanoparticle aggregates.

FIG. 9 is TEM image photograph of DNAzyme-assembled 13 mm gold nanoparticle aggregates.

FIGS. 10A-10S depict nucleic acid enzymes utilizing specific analytes as co-factors for catalytic cleavage reactions.

### DETAILED DESCRIPTION

In a related application, U.S. Ser. No. 10/144,679, filed May 10, 2002, entitled “Simple catalytic DNA biosensors for ions based on color changes,” a calorimetric sensor was disclosed that in one aspect utilized heating to speed the analyte catalyzed disaggregation of an aggregate. In this prior sensor system, a sample was added to a DNAzyme/Substrate/particle aggregate. The mixture was then heated to bring about the disaggregation of the aggregate if the sample included the selected analyte.

The present invention makes use of the discovery that the addition of invasive DNA to a DNA-RNAzyme/Substrate/particle aggregate can speed disaggregation of the aggregate without heating. In this manner, a light-up calorimetric sensor is provided that undergoes the desired color change in response to a selected analyte at room temperature, thus overcoming a disadvantage of the sensor system disclosed in U.S. Ser. No. 10/144,679.

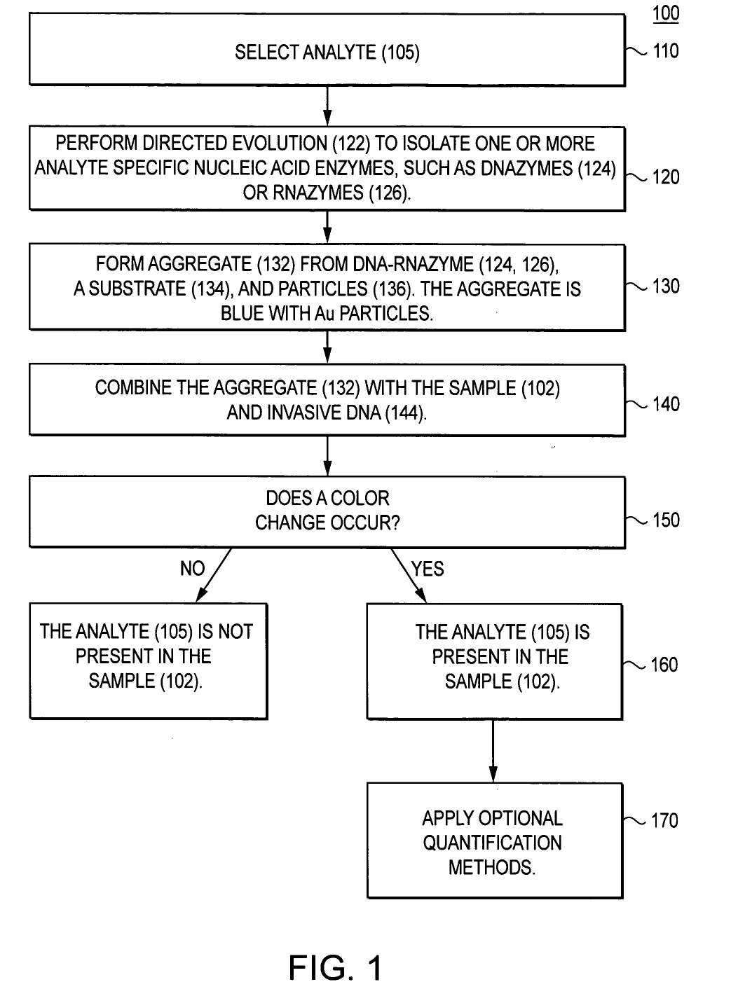

FIG. 1 represents a colorimetric analytic method 100 of determining the presence and optionally the concentration of an analyte 105 in a sample 102 (not shown). In 110, the analyte 105 for which the method 100 will determine the presence/concentration of is selected.

In one aspect, the analyte 105 may be any ion that can serve as a co-factor for a cleavage reaction, as discussed further below. Preferable monovalent metal ions having a ⁺1 formal oxidation state (I) include Li(I), Tl(I), and Ag(I). Preferable divalent metal ions having a ⁺2 formal oxidation state (II) include Mg(II), Ca(II), Mn(II), Co(II), Ni(II), Zn(II), Cd(II), Cu(II), Pb(II), Hg(II), Pt(II), Ra(II), Sr(II), Ni(II), and Ba(II). Preferable trivalent and higher metal ions having ⁺3 (III), ⁺4 (IV), ⁺5 (V), or ⁺6 (VI) formal oxidation states include Co(II), Cr(III), Ce(IV), As(V), U(VI), Cr(VI), and lanthanide ions. More preferred analyte ions include Ag(I), Pb(II), Hg(II), U(VI), and Cr(VI) due to the toxicity of these ions to living organisms. At present, and especially preferred analyte ion is Pb(II).

Once the analyte 105 is selected in 110, in 120 directed evolution 122 may be performed to isolate nucleic acid enzymes, such as DNAzyme 124 or RNAzyme 126, which will catalyze substrate cleavage in the presence of the analyte. The directed evolution 122 is preferably a type of in vitro selection method that selects molecules on the basis of their ability to interact with another constituent. Thus, the procedure of the directed evolution 122 may be selected to provide the DNA-RNAzymes that demonstrate enhanced substrate cleavage in the presence of the selected analyte 105 (thereby providing sensor sensitivity). The procedure also may be selected to exclude DNA-RNAzymes that demonstrate cleavage in the presence of selected analytes, but additionally demonstrate cleavage in the presence of non-selected analytes and/or other species present in the sample 102 (thereby providing sensor selectivity).

The directed evolution 122 may be any selection routine that provides nucleic acid enzymes that will catalyze the cleavage of a substrate in the presence of the desired analyte with the desired sensitivity and selectivity. In one aspect, the directed evolution 122 may be initiated with a DNA library that includes a large collection of strands (e.g. 10¹⁶ sequence variants), each having a different variation of bases. Phosphoramidite chemistry may be utilized to generate the strands. The DNA library is then screened for strands that bind the analyte. These strands are isolated and amplified, such as by PCR. The amplified strands may then be mutated to reintroduce variation. These strands are then screened for strands that more effectively bind the analyte. By repeating the selection, amplification, and mutation sequence while increasing the amount of binding efficiency required for selection, strands that more effectively bind the analyte, thus providing greater sensitivity, may be generated.

In one aspect, a technique referred to as in vitro selection and evolution may be utilized to perform the directed evolution 122. Details regarding this technique may be found in Breaker, R. R., Joyce, G. F., “A DNA enzyme with Mg²⁺-dependent RNA phosphoesterase activity,” Chem. Biol. 1995, 2:655-660; and in Jing Li, et al., “In Vitro Selection and Characterization of a Highly Efficient Zn(II)-dependent RNA-cleaving Deoxyribozyme,” Nucleic Acids Res. 28, 481-488 (2000).

In another aspect, nucleic acid enzymes having greater selectivity to a specific analyte may be obtained by introducing a negative selection process into the directed evolution 122. After selecting the strands having high sensitivity to the analyte, a similar selection, amplification, and mutation sequence may be applied, but to be selected, the strand must not bind closely related analytes.

For example, a DNAzyme may be selected that specifically binds Pb(II), while not significantly binding Mg(II), Ca(II), Co(II), or other competing metal ions. In one aspect, this may be achieved by isolating DNAzymes that bind Pb(II), then removing any DNAzymes that bind Mg(II), Ca(II), or Co(II). In another aspect, DNAzymes that bind Mg(II), Ca(II), or Co(II) are first discarded and then those that bind Pb(II) are isolated. In this manner, the selectivity of the DNAzyme may be increased. Details regarding one method to increase DNAzyme selectivity may be found in Bruesehoff, P. J., et al., “Improving Metal Ion Specificity During In Vitro Selection of Catalytic DNA,”Combinatorial Chemistry and High Throughput Screening, 5, 327-355 (2002).

The DNA-RNAzymes 124, 126 are nucleic acid enzymes having the ability to catalyze chemical reactions, such as hydrolytic cleavage, in the presence of a co-factor. The DNAzyme 124 includes deoxyribonucleotides, while the RNAzyme 126 includes ribonucleotides. The nucleotides from which the DNA-RNAzyme 124, 126 are formed may be natural, unnatural, or modified nucleic acids. Peptide nucleic acids (PNAs), which include a polyamide backbone and nucleoside bases (available from Biosearch, Inc., Bedford, Mass., for example), also may be useful.

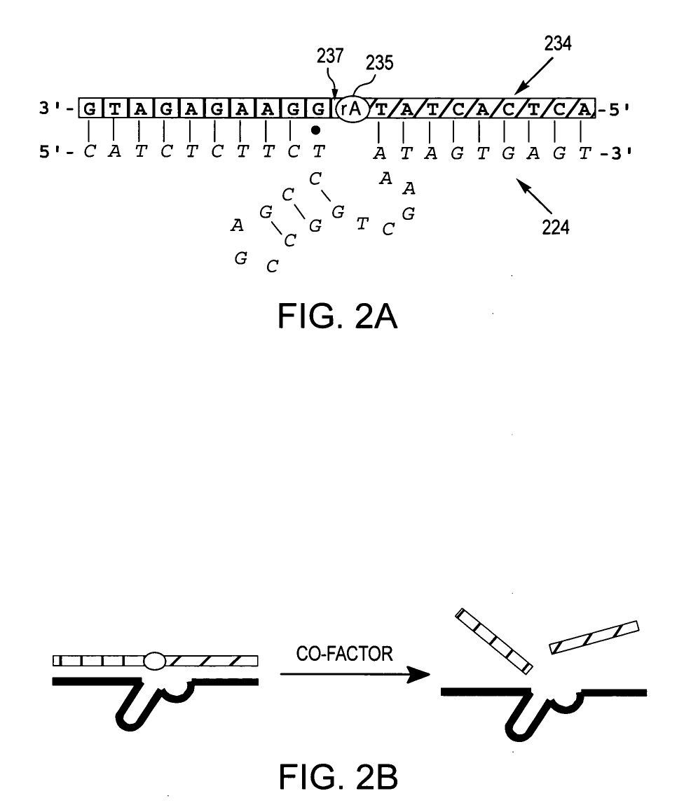

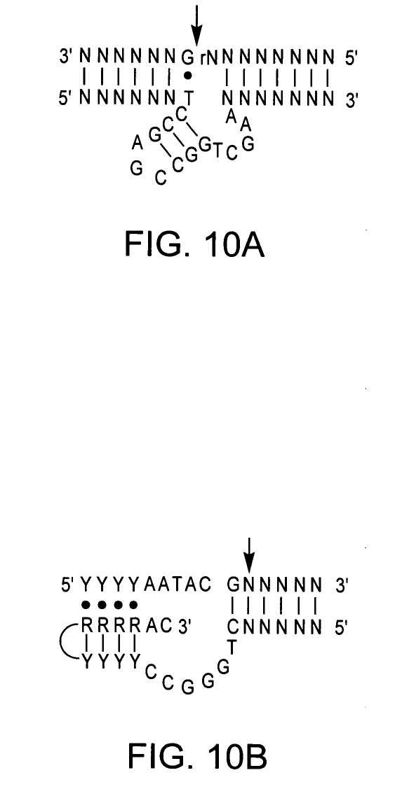

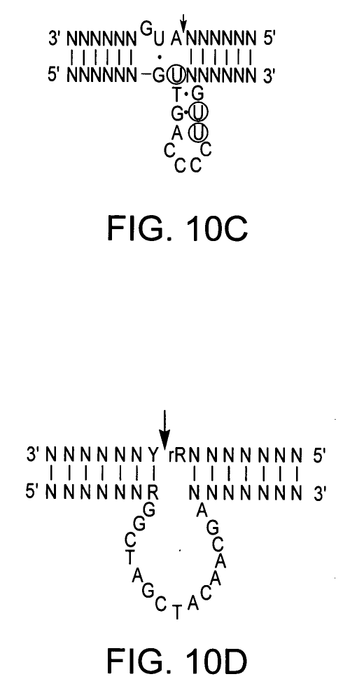

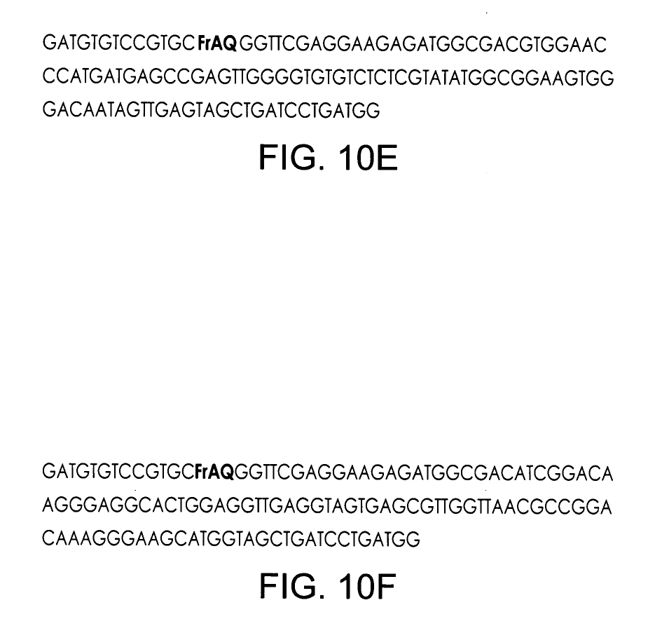

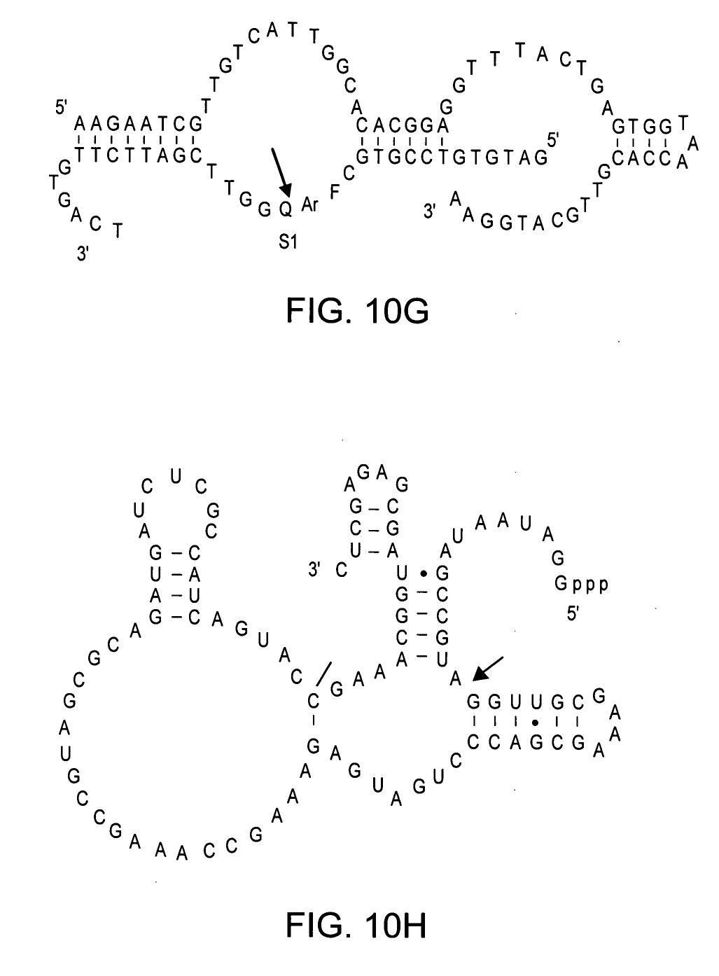

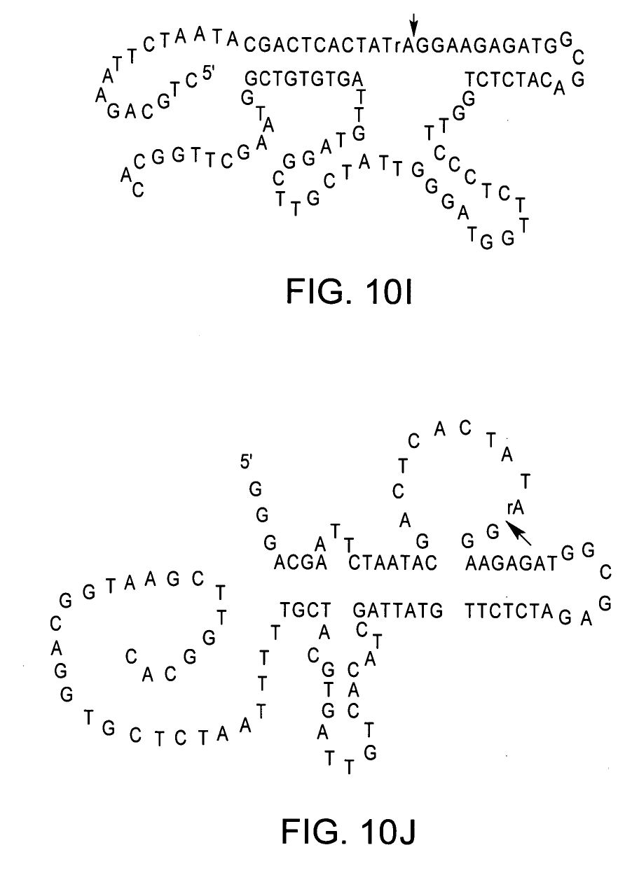

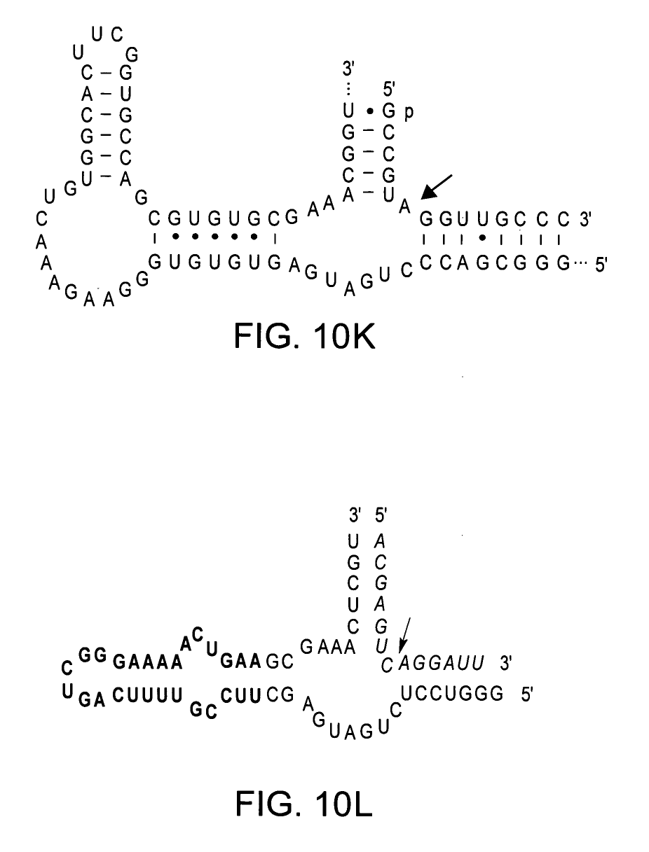

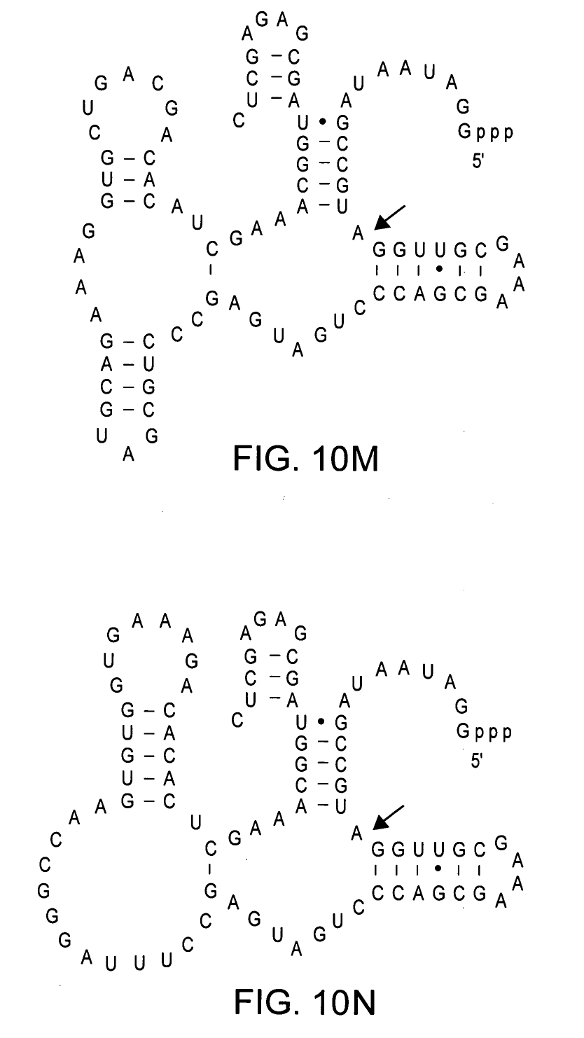

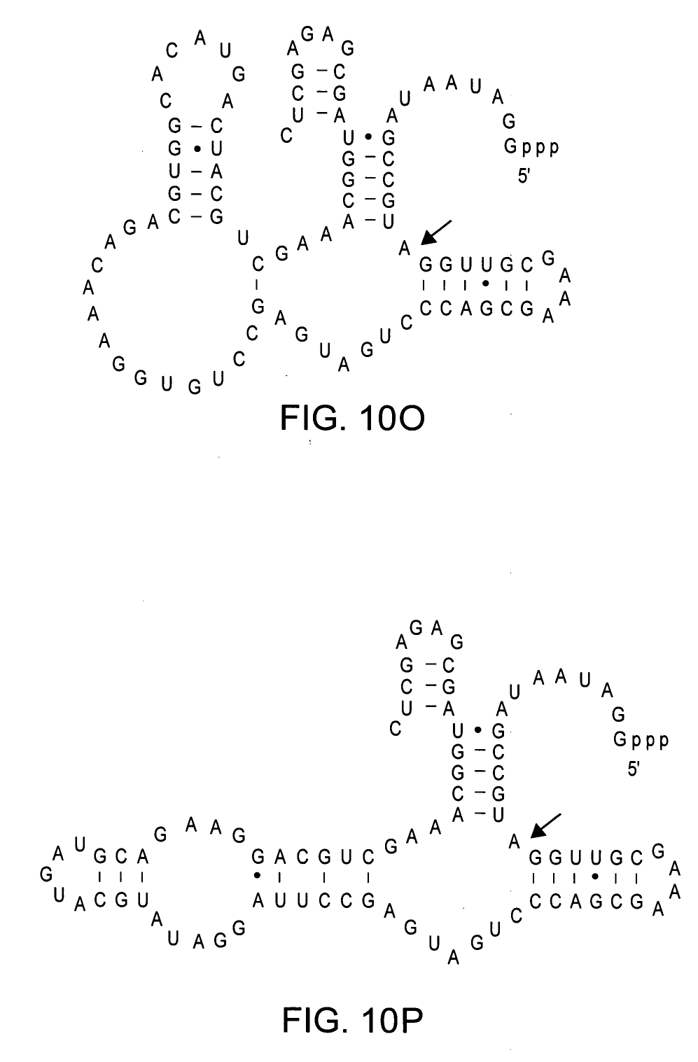

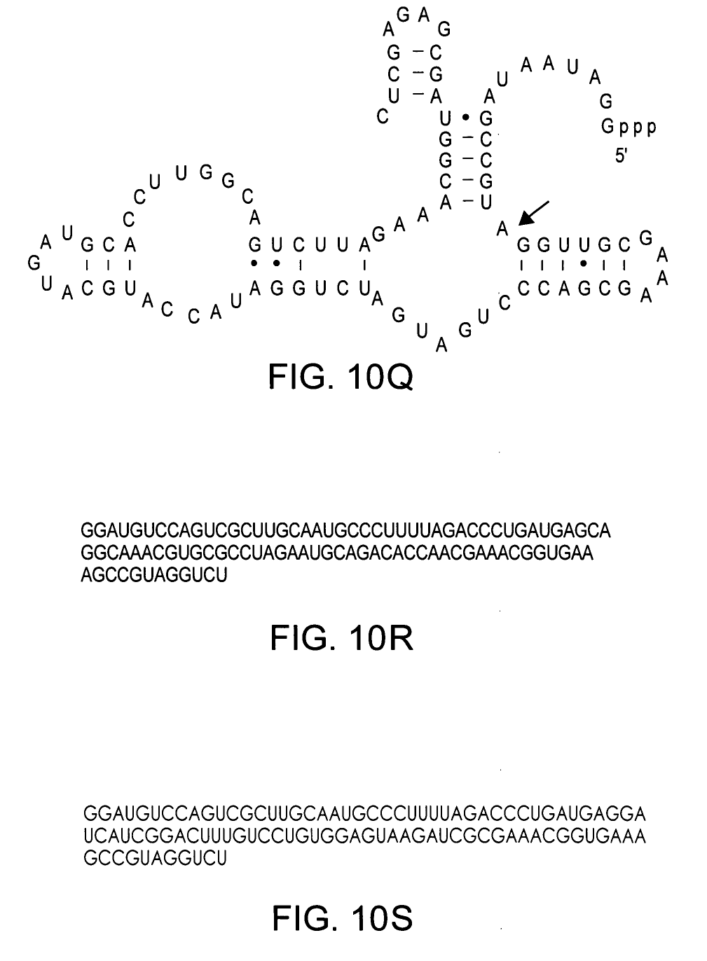

The table below lists specific analytes, the Figure in which the corresponding nucleic acid enzyme sequence that utilizes the analyte as a cleavage co-factor may be found, and the reference or references where each nucleic acid enzyme sequence is described. FIGS. 10A-10D and 10G depict trans-acting nucleic acid enzymes that are specific to metal ions having ⁺2 formal oxidation states. FIGS. 10K-10L depict trans-acting nucleic acid enzymes that also may serve as suitable nucleic acid enzymes. FIGS. 10E-10F and 10H-10-J depict cis-acting nucleic acid enzymes that are specific to metal ions having ⁺2 formal oxidation states. FIGS. 10M-10S depict cis-acting nucleic acid enzymes that also may serve as suitable nucleic acid enzymes. Preferably, cis-acting nucleic acid enzymes may be cut into two strands (truncated), such as by cleaving the GAAA loop presented at the right side of the enzymes 10M through 10Q, to provide a catalytic system. Any of these, and other, nucleic acid sequences may be adapted for use as the DNA-RNAzymes 124, 126. Trans- and cis-acting enzymes are discussed further with regard to FIG. 2A.

| | Nucleic Acid | |
|---|---|---|
| | Enzyme | |
| | Figure No. | |
| Analyte | (SEQ ID NO) | Reference/s |
| Pb(II) | | 1). Santoro, S. W.; Joyce, G. F. Proc. |
| | (SEQ ID NO: 26) | Natl. Acad. Sci. U.S.A. 1997, 94, |
| | | 4262-4266. |
| | | 2). Faulhammer, D.; Famulok, M. |
| | | Angew. Chem., Int. Ed. Engl. 1996, 35, |
| | | 2837-2841. |
| | | 3). Li, J.; Zheng, W.; Kwon, A. H.; Lu, |
| | | Y. Nucleic Acids Res. 2000, 28, |
| | | 481-488. |
| Cu(II) | | 1). Carmi, N.; Shultz, L. A.; Breaker, |
| | (SEQ ID NO: 27) | R. R. Chem. Biol. 1996, 3, 1039-1046. |
| | R, Y, and N | 2). Carmi, N.; Balkhi, H. R.; Breaker, |
| | represent purine, | R. R. Proc. Natl. Acad. Sci. U.S.A. |
| | pyrimidine, | 1998, 95, 2233-2237. |
| | and any | |
| | nucleotide, | |
| | respectively. | |
| Zn(II) | | Santoro, S. W.; Joyce, G. F.; Sakthivel, |
| | (SEQ ID NO: 28) | K.; Gramatikova, S.; Barbas, C. F., III. |
| | Circled “U” | J. Am. Chem. Soc. 2000, 122, |
| | represent | 2433-2439. |
| | C5-imidazole- | |
| | functionalized | |
| | deoxyuridine. | |
| Mg(II) | | Santoro, S. W.; Joyce, G. F. Proc. |
| | (SEQ ID NO: 29) | Natl. Acad. Sci. U.S.A. 1997, 94, |
| | | 4262-4266. |
| Mn(II) | | Liu, Z.; Mei, S. H. J.; Brennan, J. D.; |
| | (SEQ ID NO: 30) | Li, Y. J. Am. Chem. Soc. 2003, 125, |
| | | 7539-7545. |
| Mn(II) | | Liu, Z.; Mei, S. H. J.; Brennan, J. D.; |
| & | (SEQ ID NO: 31) | Li, Y. J. Am. Chem. Soc. 2003, 125, |
| Ni(II) | | 7539-7545. |
| Co(II) | | Mei, S. H. J.; Liu, Z.; Brennan, J. D.; |
| | (SEQ ID NO: 32) | Li, Y. J. Am. Chem. Soc. 2003, 125, |
| | F: fluorescein-dT | 412-420. |
| | Q: DABCYL-dT | |
| | Ar: adenine | |
| | ribonucleotide | |
| Co(II) | | Seetharaman, S.; Zivarts, M.; |
| | (SEQ ID NO: 33) | Sudarsan, N.; Breaker, R. R. Nature |
| | | Biotechnology 2001, 19, 336-341. |
| Co(II) | | Bruesehoff, P., J.; Li, J.; Augustine, I. |
| | (SEQ ID NO: 34) | A. J.; Lu, Y. Combinat. Chem. High |
| | | Throughput Screening, 2002, 5, |
| | | 327-335. |
| Zn(II) | | Bruesehoff, P., J.; Li, J.; Augustine, I. |
| | (SEQ ID NO: 35) | A. J.; Lu, Y. Combinat. Chem. High |
| | | Throughput Screening, 2002, 5, |
| | | 327-335. |
| ATP | | Tang, J.; Breaker, R. R. Chem. Biol. |
| | (SEQ ID NO: 36) | 1997, 4, 453-459. |
| HIV-1-RT | | Hartig, J. S.; Famulok, M. Angew. |
| | (SEQ ID NO: 37) | Chem., Int. Ed. Engl. 2002, 41, |
| | | 4263-4266. |
| cGMP | | Koizumi, M.; Soukup, G. A.; Kerr, J. |
| | (SEQ ID NO: 38) | N. Q.; Breaker, R. R. Nat. Struct. Biol. |
| | | 1999, 6, 1062-1071. |
| cCMP | | Koizumi, M.; Soukup, G. A.; Kerr, J. |
| | (SEQ ID NO: 39) | N. Q.; Breaker, R. R. Nat. Struct. Biol. |
| | | 1999, 6, 1062-1071. |
| cAMP | | Koizumi, M.; Soukup, G. A.; Kerr, J. |
| | (SEQ ID NO: 40) | N. Q.; Breaker, R. R. Nat. Struct. Biol. |
| | | 1999, 6, 1062-1071. |
| FMN | | Soukup, G. A.; Breaker, R. R. Proc. |
| | (SEQ ID NO: 41) | Natl. Acad. Sci. U.S.A. 1999, 96, |
| | | 3584-3589. |
| Theo | | Soukup, G. A.; Breaker, R. R. Proc. |
| | (SEQ ID NO: 42) | Natl. Acad. Sci. U.S.A. 1999, 96, |
| | | 3584-3589. |
| Aspartame | | Ferguson, A.; Boomer, R. M.; Kurz, |
| | (SEQ ID NO: 43) | M.; Keene, S. C.; Diener, J. L.; Keefe, |
| | | A. D.; Wilson, C.; Cload, S. T. |
| | | Nucleic Acids Res. 2004, 32, |
| | | 1756-1766. |
| Caffeine | | Ferguson, A.; Boomer, R. M.; Kurz, |
| | (SEQ ID NO: 44) | M.; Keene, S. C.; Diener, J. L.; Keefe, |
| | | A. D.; Wilson, C.; Cload, S. T. |
| | | Nucleic Acids Res. 2004, 32, |
| | | 1756-1766. |

While both DNAzymes and RNAzymes can form duplexes with a DNA-based substrate, such as substrate 134 discussed below, the RNAzyme/Substrate duplex may be less stable than the DNAzyme/Substrate duplex. Additionally, DNAzymes are easier to synthesize and more robust than their RNAzyme counterparts.

The deoxyribonulceotides of the DNAzyme 124 and the complementary substrate strand 134 may be substituted with their corresponding ribonucleotides, thus providing the RNAzyme 126. For example, one or more ribo-cytosines may be substituted for the cytosines, one or more ribo-guanines may be substituted for the guanines, one or more ribo-adenosines may be substituted for the adenosines, and one or more uracils may be substituted for the thymines. In this manner, nucleic acid enzymes including DNA bases, RNA bases, or both may independently hybridize with complementary substrate strands that include DNA bases, RNA bases, or both.

After selecting an appropriate nucleic acid enzyme or enzymes in 120, an aggregate 132 may be formed in 130. The aggregate 132 includes the nucleic acid enzymes; the substrate 134; and oligonucleotide functionalized particles 136. Considering the physical size of its components, the aggregate 132 may be quite large. In fact, transmission electron microscopy (TEM) studies suggest that individual aggregates may range from 100 nm to 1 micron, and may agglomerate to form larger structures.

The substrate 134 may be any oligonucleotide that may hybridize with and be cleaved by the nucleic acid enzyme in the presence of the analyte 105. The oligonucleotide may be modified with a cleavage species, which allows cleavage of the substrate into two fragments by the nucleic acid enzyme. In one aspect, the substrate 134 is a strand complementary to the nucleic acid enzyme and may be extended to form a 12-mer overhang on each end to hybridize with the oligonucleotide functionalized particles 136. For example, if an oligonucleotide functionalized particle had a base sequence of 5′-CACGAGTTGACA, an appropriate overhang sequence for the substrate could be 3′-GTGCTCAACTGT.

Because the particles 136 demonstrate distance-dependent optical properties, the particles are one color when closely held in the aggregate 132 and undergo a color change as the distance between the particles increases. For example, when the particles 136 are gold nanoparticles, the aggregate 132 displays a blue color in aqueous solution that turns red as disaggregation proceeds. Disaggregation occurs when the substrate 134 that holds the functionalized particles 136 together is cleaved, thereby allowing the particles to separate from the aggregate 132. Thus, as the particles 136 diffuse away from the aggregate 132, the solution changes from blue to red.

The particles 136 may be any species that demonstrate distance-dependent optical properties and are compatible with the operation of the sensor system. Suitable particles may include metals, such as gold, silver, copper, and platinum; semiconductors, such as CdSe, CdS, and CdS or CdSe coated with ZnS; and magnetic colloidal materials, such as those described in Josephson, Lee, et al., Angewandte Chemie, International Edition (2001), 40(17), 3204-3206. Specific useful particles may include ZnS, ZnO, TiO₂, Agl, AgBr, Hgl₂, PbS, PbSe, ZnTe, CdTe, In₂S₃, In₂Se₃, Cd₃P₂, Cd₃As₂, InAs, and GaAs.

In a preferred aspect, the particles are gold (Au) nanoparticles and have an average diameter from 5 to 70 nanometers (nm) or from 10 to 50 nm. In an aspect especially preferred at present, gold nanoparticles having an average diameter of from 10 to 15 nm are functionalized to the oligonucleotides.

For a more detailed treatment of how to prepare gold functionalized oligonucleotides, See U.S. Pat. No. 6,361,944; Mirkin, et al., Nature (London) 1996, 382, 607-609; Storhoff, et al., J. Am. Chem. Soc. 1998, 20, 1959-1064; and Storhoff, et al., Chem. Rev. (Washington, D.C.) 1999, 99, 1849-1862. While gold nanoparticles are presently preferred, other fluorophores, such as dyes, inorganic crystals, quantum dots, and the like that undergo a distance-dependent color change also may be attached to oligonucleotides and utilized.

In 140 the aggregate 132 from 130 may be combined with the sample 102 and invasive DNA 144. In 150 the sample 102 is monitored for a color change. If a color change does not occur, then the analyte 105 is not present in the sample 102. If a color change does occur in 160, the analyte 105 is present in the sample 102. Thus, the analytic method 100 provides a “light-up” sensor system because a color change occurs in the presence of the analyte 105.

The color change signifies that the analyte 105 is an appropriate co-factor to catalyze cleavage of the substrate 134, which is hybridized with the oligonucleotide functionalized particles 136. This cleavage is believed to cause the substrate 134 to split into two fragments, thus allowing the particles 136 to diffuse away from the aggregate 132 and into the solution of the sample 102. While this cleavage of the substrate 134 is believed to proceed at room temperature, it is thought that a significant portion of the 9 base pairs forming each cleaved portion of the substrate remain hybridized with the nucleic acid enzyme. Thus, for disaggregation to occur, it is preferable to disrupt this hybridization.

The invasive DNA 144 is believed to “invade” the aggregate 132 and assist in releasing of the cleaved substrate fragments. While not wishing to be bound by any particular theory, it is believed that equilibrium forces causes a competition for the sites on the cleaved portions of the substrate 134 to occur between the nucleic acid enzyme and the invasive DNA 144. Because this equilibrium favors hybridization of the substrate 134 with the invasive DNA 144, the cleaved portions of the substrate 134 are pulled away from the nucleic acid enzyme, thus speeding disaggregation. As the cleaved portions of the substrate 134 hybridize with the invasive DNA 144, the attached particles 136 diffuse away from the aggregate 132 and provide the desired color change. Although the term “invasive DNA” is used throughout this specification and appended claims for consistency, if the substrate 134 includes ribonucleotides, the invasive DNA 144 also may include ribonucleotides.

While the invasive DNA 144 may be any oligonucleotide that is at least partially complementary to the cleaved fragments of the substrate 134, preferably, the invasive DNA 144 includes relatively short pieces of DNA. In one aspect, the invasive DNA 144 includes at least two types of DNA strands, each being at least partially complementary to one of the two cleaved substrate fragments. In another aspect, at least one terminal base of each of the cleaved substrate fragments is complementary to at least one terminal base of each of the invasive DNA strands. In yet another aspect, the invasive DNA 144 includes at least two types of DNA strands, each being fully complementary to one of the two cleaved substrate fragments. In yet another aspect, the invasive DNA 144 has from 2 to 10 or from 4 to 8, which includes 2, 4, 6, or 8 fewer bases capable of hybridizing with the corresponding cleaved substrate fragment. At present, especially preferred invasive DNA strands have 6 fewer complementary bases than the corresponding cleaved substrate fragment.

The degree the color changes in response to the analyte 105 may be quantified by colorimetric quantification methods known to those of ordinary skill in the art in 170. Various color comparator wheels, such as those available from Hach Co., Loveland, Colo. or LaMotte Co., Chestertown, Md. may be adapted for use with the present invention. Standard samples containing known amounts of the selected analyte may be analyzed in addition to the test sample to increase the accuracy of the comparison. If higher precision is desired, various types of spectrophotometers may be used to plot a Beer's curve in the desired concentration range. The color of the test sample may then be compared with the curve and the concentration of the analyte present in the test sample determined. Suitable spectrophotometers include the Hewlett-Packard 8453 and the Bausch & Lomb Spec-20.

In yet another aspect, the method 100 may be modified to determine the sensitivity and selectivity of an endonuclease, such as a nucleic acid enzyme, for detecting the analyte 105. In this aspect, an aggregate is formed from the substrate 134 and the particles 136, but without the DNA-RNAzymes 124, 126 in 130. This aggregate is combined with the analyte of interest and the invasive DNA in 140. The endonuclease, such as one created by the directed evolution 122, is then added. If the endonuclease can cleave the substrate 134 with the desired sensitivity and selectivity in the presence of the analyte 105, the endonuclease may be used to analyze for the analyte 105 in a calorimetric sensor system. In this aspect, the endonuclease or nucleic acid enzyme also may be considered an analyte. In this manner, multiple endonucleases generated from the directed evolution 122 may be tested for use in a calorimetric sensor system.

FIG. 2A depicts a DNAzyme 224 that depends on Pb(II) as a co-factor to display catalytic activity. As may be seen from the base pairs, the DNAzyme 224 may hybridize to a complementary substrate strand 234 that includes a cleavage species, such as ribo-adenosine 235. Other than the ribo-adenosine 235 cleavage species, the depicted complementary substrate strand 234 is formed from deoxyribonucleosides. While one base sequence for the DNAzyme and the complementary substrate strand are shown, the bases may be changed on both strands to maintain the pairings. For example, any C on either strand may be changed to T, as long as the paired base is changed from G to A.

The base pairing regions of the DNAzyme 224 and the complementary substrate strand 234 may be extended or truncated, as long as sufficient bases exist to maintain the desired cleavage of the substrate. While many modifications to the enzyme and substrate are possible, modifications made to the catalytic core region of the enzyme can have significant effects on the catalytic efficiency or analyte specificity of the enzyme. A more detailed discussion of such modifications and the resulting effects on catalytic activity may be found in Brown, A., et al., “A Lead-dependent DNAzyme with a Two-Step Mechanism,” Biochemistry, 42, 7152-7161 (2003).

The ribo-adenosine (rA) 235 provides a cleavage site 237, where the DNAzyme 224 is believed to hydrolytically cleave the substrate 234 in the presence of the co-factor, as depicted in FIG. 2B. This cleavage reaction results in the substrate 234 being split into its 3′ and 5′ fragments as depicted in FIG. 2B. In addition to the ribo-adenosine 235, the cleavage species utilized with a DNAzyme, such as the DNAzyme 224, also may include ribo-cytosine (rC), ribo-guanine (rG), and Uracil (U). Similarly, if the nucleic acid enzyme were a RNAzyme (not shown) appropriate cleavage species also may include rA, rC, rG, and U.

The DNAzyme 224 and the complementary substrate strand 234 may be separate strands, as depicted in FIG. 2A, or the DNAzyme and the substrate may be part of the same nucleic acid strand. When the DNAzyme and the complementary substrate are different nucleic acid strands, the DNAzyme may be referred to as a “trans-acting enzyme.” Trans-acting enzymes have the advantage of being able to cleave multiple complementary substrates. If the DNAzyme and the complementary substrate are part of the same nucleic acid strand, such as depicted in FIG. 10E for example, the DNAzyme may be referred to as a “cis-acting enzyme.”

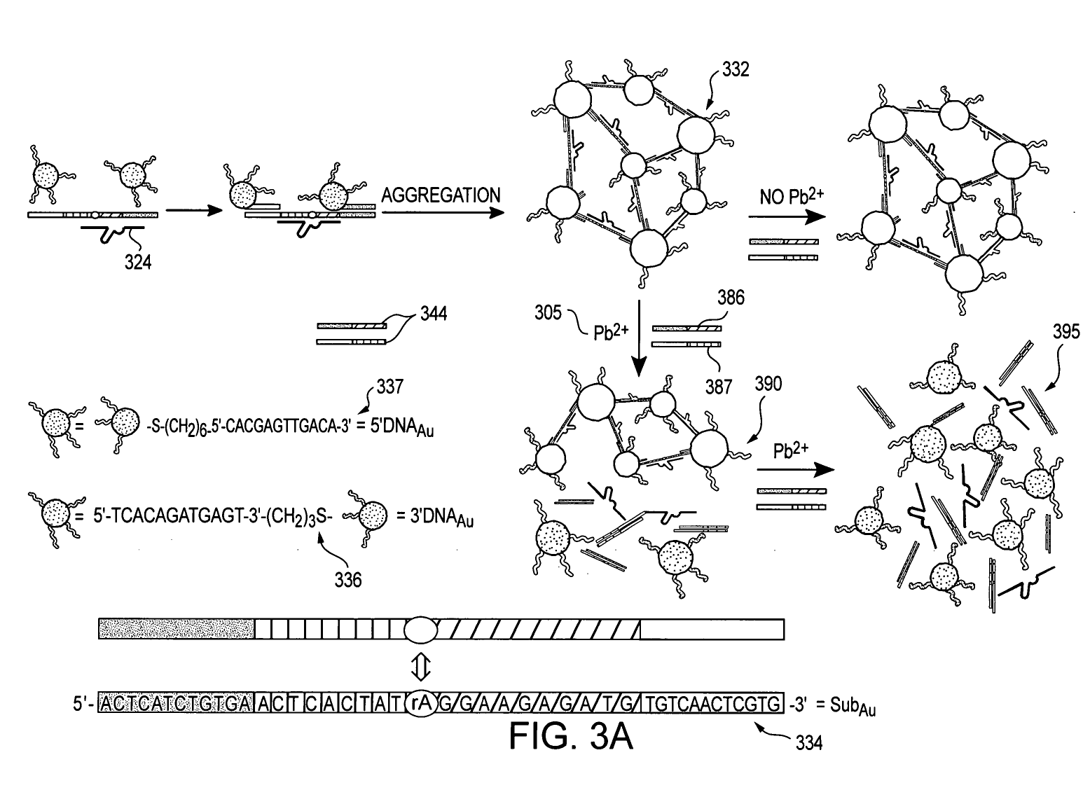

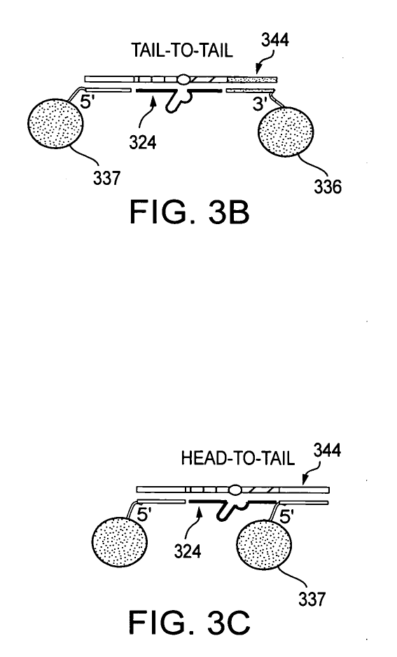

FIG. 3A depicts the disaggregation of an aggregate 332 in the presence of a Pb(II) analyte 305 and invasive DNA 344. The aggregate 332 is formed from a DNAzyme 324 and a substrate strand 334, which is hybridized to 3′ and 5′ thiol-oligonucleotide functionalized particles 336 and 337, respectively. The substrate strand 334 was extended on both the 3′- and 5′-ends for 12 bases, allowing hybridization with the 12-mer DNA functionalized particles 336, 337. The catalytic core of the DNAzyme 324 includes the “8-17” DNAzyme motif, which exhibits high activity in the presence of the Pb(II) cation.

The invasive DNA 344 includes a 3′ strand 387 and a 5′ strand 386. In the presence of the analyte 305 and the invasive strands 386 and 387, the blue aggregate 332 begins to disaggregate to form partial aggregate 390. This partial disaggregation adds red color to the blue solution as the particles diffuse away from the aggregate 332, thus giving a purple solution. If enough of the analyte 305 is present in the sample, the reaction will continue until the aggregate 332 is completely disaggregated, to give 395. This results in a red solution due to the greater distance between the nanoparticles.

The alignment of the particles (tail-to-tail or head-to-tail) with respect to each other may influence how tightly the moieties that form the aggregate bind together. FIGS. 3A and 3B depict that the aggregate 332 may be formed from the DNAzyme 324 and the substrate strand 344 where the functionalized particles, such as 336 and 337, hybridize in a tail-to-tail (FIG. 3B) arrangement with the substrate strand 344. Tail-to-tail or head-to-tail (FIG. 3C) hybridization may be selected by reversing the end of the oligonucleotide to which the particle is attached. Thus, a head-to-tail alignment may be selected through the use of a single thiol-modified DNA strand, such as 337, while for tail-to-tail alignment both the 3′- and 5′-thiol-modified DNA strands may be coupled to the particles.

At present, the tail-to-tail hybridization arrangement of FIGS. 3A and 3B is preferred because the head-to-tail hybridization arrangement of FIG. 3C may produce aggregates that sterically hinder the catalytic activity of the DNAzyme. However, this steric hindrance may be reduced through a reduction in the average diameter of the particles or through the use of a longer substrate, for example.

The ionic strength of the sample may influence how tightly the moieties that form the aggregate bind together. Higher salt concentrations favor aggregation, thus slowing sensor response, while lower salt concentrations may lack the ionic strength necessary to maintain the aggregates. In one aspect, the sample may include or be modified with a reagent to include a monovalent metal ion concentration of 30 mM and greater. The ionic strength of the sample may be modified with Na⁺ ions, for example. In a preferred aspect, the monovalent metal ion concentration of the sample, which contains the aggregate, is from 28 to 40 mM. At present, an especially preferred monovalent metal ion concentration is about 30 mM. pH also may influence the aggregate binding, possibly attributable to the protonation of the polynucleotide base pairs at lower pH. In one aspect, an approximately neutral pH is preferred.

Thus, the performance of the sensor may be improved by adjusting the ionic strength and pH of the sample prior to combining it with the aggregate. Depending on the sample, it may be preferable to add the sample or analyte to a solution containing the aggregate (where the ionic strength and pH may be controlled) or the reverse.

The sensor system, including the substrate, oligonucleotide functionalized particles, and invasive DNA may be provided in the form of a kit. In one aspect, the kit includes the desired analyte specific endonuclease or nucleic acid enzyme that is at least partially complementary to the substrate. In yet another aspect, the kit excludes the endonuclease/nucleic acid enzyme, which is then provided by the user or provided separately. In this aspect, the kit also may be used to determine the specificity and/or selectivity of various endonucleases to a selected analyte. Thus, the kit may be used to select an appropriate endonuclease in addition to detecting the analyte. In yet another aspect, the kit includes an exterior package that encloses a DNAzyme, a complementary substrate, oligonucleotide functionalized particles, and invasive DNA.

One or more of these components may be separated into individual containers, or they may be provided in their aggregated state. If separated, the aggregate may be formed before introducing the sample. The invasive DNA may be held in a separate container so it may be added to the sample prior to combination with the aggregate. Additional buffers and/or pH modifiers may be provided in the kit to adjust the ionic strength and/or pH of the sample.

The containers may take the form of bottles, tubs, sachets, envelopes, tubes, ampoules, and the like, which may be formed in part or in whole from plastic, glass, paper, foil, MYLAR®, wax, and the like. The containers may be equipped with fully or partially detachable lids that may initially be part of the containers or may be affixed to the containers by mechanical, adhesive, or other means. The containers also may be equipped with stoppers, allowing access to the contents by syringe needle. In one aspect, the exterior package may be made of paper or plastic, while the containers are glass ampoules.

The exterior package may include instructions regarding the use of the components. Color comparators; standard analyte solutions, such as a 10 μm solution of the analyte; and visualization aids, such as thin layer chromatography (TLC) plates, test tubes, and cuvettes, also may be included. Containers having two or more compartments separated by a membrane that may be removed to allow mixing may be included. The exterior package also may include filters and dilution reagents that allow preparation of the sample for analysis.

In another aspect, in addition to the sensor system of the present invention, the kit also may include multiple sensor systems to further increase the reliability of analyte determination and reduce the probability of user error. In one aspect, multiple light-up sensor systems in accord with the present invention may be included. In another aspect, a “light-down” sensor system may be included with the light-up sensor system of the present invention.

The presently claimed sensor system may be considered a light-up sensor because a color change occurs (blue to red) in the presence of the analyte. Conversely, in a light-down sensor system, a color change is not observed in the presence of the analyte. Thus, a light-up system may give a false result by lighting up when the analyte is absent, while a light-down sensor system may not undergo a color change when the analyte is present. Combining a sensor system using light-down chemistry with the presently claimed light-up sensor may reduce the probability of an inaccurate analyte determination.

Suitable light-down sensors for inclusion in the presently claimed kit may rely on DNAzyme/Substrate/particle aggregates that are not formed in the presence of the selected analyte. Thus, for these sensors, a color change from aggregate formation is observed when the selected analyte is not present in the sample. In one aspect, these light-down sensors may rely on a tail-to-tail particle arrangement coupled with nanoparticles having average diameters of about 43 nm to provide aggregation at room temperature in the absence of the analyte. A more detailed description of suitable light-down sensor systems for inclusion in the presently claimed kit may be found, for example, in U.S. patent application Ser. No. 10/756,825, filed Jan. 13, 2004, entitled “Biosensors Based on Directed Assembly of Particles,” which is hereby incorporated by reference.

The preceding description is not intended to limit the scope of the invention to the preferred embodiments described, but rather to enable a person of ordinary skill in the art to make and use the invention. Similarly, the examples below are not to be construed as limiting the scope of the appended claims or their equivalents, and are provided solely for illustration. It is to be understood that numerous variations can be made to the procedures below, which lie within the scope of the appended claims and their equivalents.

### EXAMPLES

All DNA samples were purchased from Integrated DNA Technology Inc., Coralville, Iowa. The substrates and enzyme portions of the DNAzyme were purified by HPLC prior to use. Gold nanoparticles having an average diameter of 13 nm were prepared and functionalized with 12-mer thiol-modified DNA following literature procedures, such as those disclosed in Storhoff, J., et al., “One-pot calorimetric differentiation of polynucleotides with single base imperfections using gold particle probes,” JACS 120: 1959-1964 (1998), for example. The average diameter of the gold nanoparticles was verified by transmission electronic microscope (JEOL 2010).

### Example 1

### Formation of the Blue Aggregate

The enzyme (17E, 400 nM), substrate (35Sub_{Au}, 100 nM), 3′DNA_{Au} (6 nM), and 5′DNA_{Au} (6 nM) were mixed with a 25 mM Tris acetate buffer, pH 8.2, 300 mM NaCl. The mixture (usually in 1 mL volume) was heated at 65° C. for 3 minutes and allowed to cool slowly to room temperature for approximately 4 hours. Blue-colored nanoparticle aggregates formed and precipitated. Optionally, the aggregates were further precipitated with a bench-top centrifuge and the supernatant was removed. The precipitated aggregates were washed three times with a buffer containing 100 mM NaCl and 25 mM tris acetate (pH 8.2) and re-dispersed in 200 μL of fresh 25 mM Tris acetate buffer, but with 100 mM NaCl.

The concentration of the aggregates in this undiluted mixture was standardized by adding 10 μL of the aggregate containing mixture to 80 μL of deionized water to disperse the aggregates. The extinction of this 9× diluted mixture was then measured at 522 nm. From this measurement, the amount of buffer solution required to provide an extinction value of 1 at 522 nm was calculated for the undiluted mixture. The appropriate amount of buffer containing 100 mM of NaCl was then added to the undiluted mixture. In this manner, the aggregate concentration in the buffer solution was adjusted so that when disaggregated; the mixture would provide an extinction of ˜9 or of ˜1 after 9 dilutions at 522 nm.

The sequences of the 17E DNAzyme (SEQ ID NO: 1) and the 17DS substrate (SEQ ID NO: 2; r denotes a single ribonucleotide) extended on each end with 12 bases 35Sub_{Au} (SEQ ID NO: 3; r denotes a single ribonucleotide) to hybridize to the 5′DNA_{Au} (SEQ ID NO: 4) and 3′DNA_{Au} (SEQ ID NO: 5) oligonucleotide functionalized gold nanoparticles are given in Table 1 below.

TABLE 1

| | | |
|---|---|---|
| | | SEQ |
| Name | Sequences | ID |

| | | |
|---|---|---|
| 17E | CAT CTC TTC TCC GAG CCG GTC GAA ATA GTG | 1 |
| | AGT | |
| 17DS | ACTCACTATrAGGAAGAGATG | 2 |
| 35Sub_{Au} | ACTCATCTGTGAACTCACTATrA GGAAGAGATGTGTCA | 3 |
| | ACTCGTG | |
| 5′DNA_{AU} | CACGAGTTGACA | 4 |
| 3′DNA_{AU} | TCACAGATGAGT | 5 |

All sequences are listed from 5′ to 3′.
rA denotes the cleavage site of the substrate (35Sub_{Au}), with r being a single nucleotide.
For 5′DNA_{AU} and 3′DNA_{AU}, a gold nanoparticle was attached via a 5′- and 3′-end thiol-linkage, respectively.

### Example 2

### Addition of Analyte and Invasive DNA

An 80 μL solution including 21 mM NaCl, 25 mM tris acetate (pH 8.2), 2.25 μM invasive DNA, and a Pb(OAc)₂ concentration 12.5% higher than desired in the test sample was combined with 10 μL of the 100 mM NaCl solution containing the aggregates from Example 1. The resultant test sample had concentrations of 30 mM NaCl, 2 μM invasive DNA, and the desired Pb(II) concentration. The color change of the solution was determined after about 5 minutes at ˜22° C.

### Example 3

### Monitoring the Performance of the Sensor

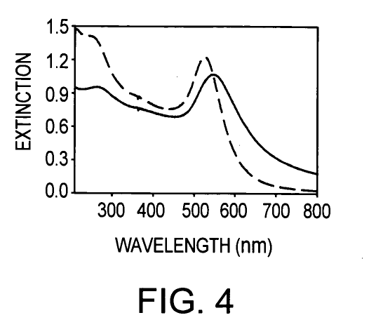

The color change of the sample from Example 2 was monitored by UV-vis extinction spectroscopy. FIG. 4 is a graph relating the extinction ratios provided at specific wavelengths from a sample during disaggregation. The dashed line in FIG. 4 shows the strong extinction peak at 522 nm exhibited by separated 13 nm nanoparticles, which provide a deep red color. As may be seen from the solid line in FIG. 4, upon aggregation, the 522 nm peak decreases in intensity and shifts to longer wavelength, while the extinction at 700 nm region increases, resulting in a red-to-blue color transition. Therefore a higher extinction ratio at 522 to 700 nm is associated with the red color of separated nanoparticles, while a low extinction ratio is associated with the blue color of aggregated nanoparticles. This extinction ratio was used to monitor the aggregation state of nanoparticles.

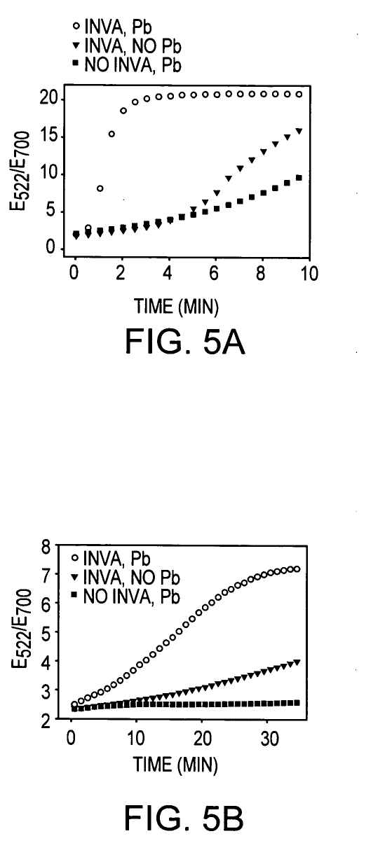

FIG. 5A is a graph depicting the change in extinction ratios over time for samples containing invasive DNA (Inva) and Pb(II) (∘), invasive DNA (Inva) without Pb(II) (▴), and a control sample containing Pb(II) without invasive DNA (▪). FIG. 5B is a similar graph utilizing the Inva-A strands in place of the Inva strands. The extinction ratio increased quickly with time for the invasive DNA/Pb(II) samples, indicating a rapid color change from blue to red and presence of the Pb(II) analyte. For the invasive DNA only samples, a color change from blue to red occurred, however, at a slower rate indicated by the slower increase in the extinction ratio. This test established that an undesirable color change could be generated by either the Inva or the Inva-A invasive DNA strands alone. Thus, too “invasive” of a DNA may bring about the disaggregation of the aggregate without the analyte (co-factor), which can result in a false positive or an undesirable background level of color change. The control, lacking invasive DNA and Pb(II), showed a very slow increase in the extinction ratio, indicating little color change.

These experiments demonstrated the utility of the sensor system to detect an analyte, but suggested that selection of the appropriate invasiveness of the invasive DNA could provide a sensor with decreased background color change and a decreased propensity to give false positives.

### Example 4

### Refining the Invasiveness of the Invasive DNA

To find a less invasive DNA, a series of invasive DNA strands having a reduced number of base-pairings with the cleaved fragments of the DNA substrate were tested as follows. A quarts UV-vis spectrophotometer cell (Hellma, Germany) was prepared as a blank by combining 60.3 μL of 25 mM tris acetate (pH 8.2), 17 μL of 100 mM NaCl-25 mM tris acetate (pH 8.2), 1.8 μL of 0.1 mM invasive DNA, and 1 μL of 1 mM Pb(OAc)₂. After a baseline measurement, 10 μL of the aggregate mixture from Example 1 was added to the cell. This addition gave a final NaCl concentration of 30 mM and a final invasive DNA concentration of 2 μM for each DNA strand. The final Pb(II) concentration was 10 μM. Samples without the Pb(II) analyte were similarly prepared, except 61.3 μL instead of 60.3 μL of the 25 mM tris acetate (pH 8.2) buffer was added to make up the sample volume.

The preferred reduced base-pairing invasive DNA strands that were prepared as outlined above are listed as Inva-2 (SEQ ID NO: 8 and SEQ ID NO: 9, left to right, respectively), Inva-4 (SEQ ID NO: 10 and SEQ ID NO: 11), Inva-6 (SEQ ID NO: 12 and SEQ ID NO: 13), and Inva-8 (SEQ ID NO: 14 and SEQ ID NO: 15) in Table 2 below. Inva refers to the 22-mer invasive DNA strands (SEQ ID NO: 6 and SEQ ID NO: 7) utilized to generate the data for FIG. 5A. The initial Inva strands underlying the preferred sequences are fully complementary to the cleaved fragments of the substrate.

TABLE 2

| | | |
|---|---|---|
| | | SEQ |
| Name | Preferred Sequences | ID |

| | | | |
|---|---|---|---|
| Inva | CACGAGTTGACACATCTCTTCC | TATAGTGAGTTCACAGATGAG | 6 & 7 |
| Inva-2 | CGAGTTGACACATCTCTTCC | TATAGTGAGTTCACAGATGAT | 8 & 9 |
| Inva-4 | AGTTGACACATCTCTTCC | TATAGTGAGTTCACAGAT | 10 & 11 |
| Inva-6 | TTGACACATCTCTTCC | TATAGTGAGTTCACAG | 12 & 13 |
| Inva-8 | GACACATCTCTTCC | TATAGTGAGTTCAC | 14 & 15 |

From “Inva” to “Inva-8”, each invasive DNA contains two DNA strands, each being at least partially complementary to one of the two cleaved portions of the substrate, respectively. Inva-6 (highlighted) was selected for the sensor design due to the rapid color response and low background increase.

Additional reduced base-pairing invasive strands that were tested are listed as Inva-2A (SEQ ID NO: 18 and SEQ ID NO: 19, left to right, respectively), Inva-4A (SEQ ID NO: 20 and SEQ ID NO: 21), Inva-6A (SEQ ID NO: 22 and SEQ ID NO: 23), and Inva-8A (SEQ ID NO: 24 and SEQ ID NO: 25) in Table 3 below. Inva-A refers to the 23 and 21-mer invasive DNA strands (SEQ ID NO: 16 and SEQ ID NO: 17) utilized in Example 3 and to generate the data for FIG. 5B. The initial Inva-A strands underlying the additional sequences are partially complementary to the cleaved fragments of the substrate, with each strand being “offset” by one base. Thus, the 23-mer invasive Inva-A strand includes one “extra” base, while the 21-mer invasive Inva-A strand includes one less base than the cleaved substrate fragments. In this manner, a “mismatch” is created between the invasive Inva-A strands and the cleaved substrate fragments.

TABLE 3

| | | |
|---|---|---|
| | | SEQ |
| Name | Additional Sequences | ID |

| | | | |
|---|---|---|---|
| Inva-A | CACGAGTTGACACATCTCTTCCT | ATAGTGAGTTCACAGATGAGT | 16 & 17 |
| Inva-2A | CGAGTTGACACATCTCTTCCT | ATAGTGAGTTCACAGATGA | 18 & 19 |
| Inva-4A | AGTTGACACATCTCTTCCT | ATAGTGAGTTCACAGAT | 20 & 21 |
| Inva-6A | TTGACACATCTCTTCCT | ATAGTGAGTTCACAG | 22 & 23 |
| Inva-8A | GACACATCTCTTCCT | ATAGTGAGTTCAC | 24 & 25 |

From “Inva-A” to “Inva-8A”, each invasive DNA contains two DNA strands, each being at least partially complementary to one of the two cleaved portions of the substrate, respectively. Of these additional sequences, Inva-4A (highlighted) had the best combination of color response and low background increase.

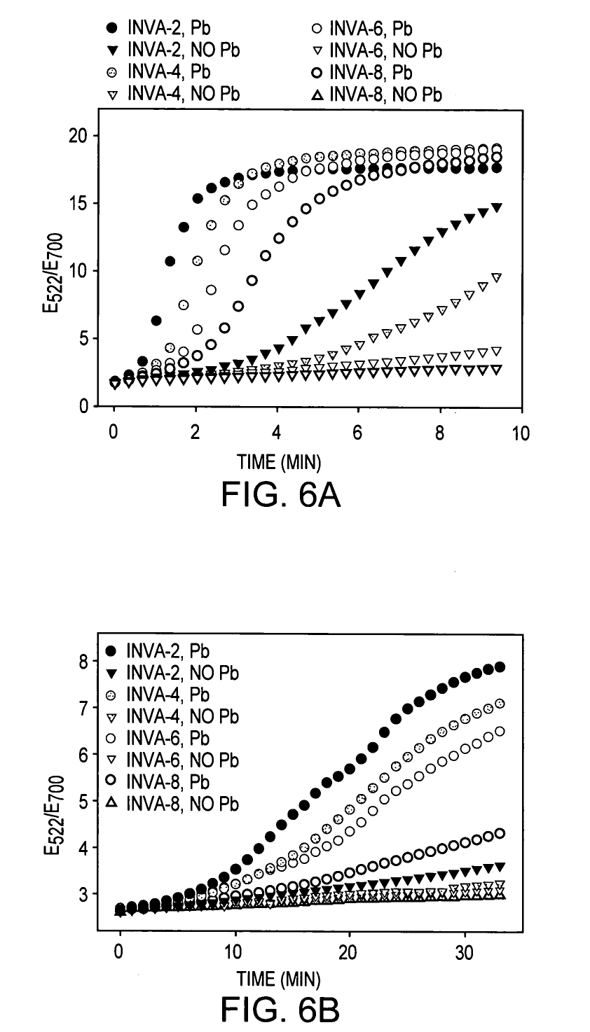

FIGS. 6A and 6B are graphs plotting the change in extinction ratios as a function of time for each of the shortened (in relation to the original Inva or Inva-A strands) invasive DNA strands with and without the Pb(II) analyte. As the strands shortened and the number of base pairings with the cleaved portions of the substrate diminished, the rate of color change in the absence of the Pb(II) analyte decreased. The rate of color change was always faster in the presence of the Pb(II) analyte with the same invasive DNA, thus establishing the ability of the sensor to detect the analyte.

The preferred Inva DNA sequences are fully complementary to each of the two fragments of the cleaved substrate, while the additional Inva-A DNA sequences are partially complementary, being mismatched by one base. While reducing the number of base-pairings for either the fully complementary Inva or the mismatched Inva-A strands decreased the overall invasiveness of the DNA and provided a desirable reduction in the level of background color change without the analyte, the reduced base Inva-6 strands maintained a rapid rate of disaggregation in the presence of the analyte.

Thus, the Inva-6 strands, having 6 fewer bases than the cleaved portions of the substrate, were chosen as the best compromise between the rate of color change in response to the analyte and the level of background color change attributable to invasive DNA only disaggregation. For these reasons, the Inva-6 strands were used to test the sensitivity and selectivity of the sensor.

While not wishing to be bound by any particular theory it is believed that the number of complementary base-pairings has a greater effect on invasiveness (thermodynamic control), while the rate of disaggregation is more strongly dependant on the ability of the ends of the cleaved substrate fragments to initially hybridize with the invasive DNA strands (kinetic control). By altering these parameters of the invasive DNA, the background levels and rate of the color change may be optimized for a specific DNA-RNAzyme and/or analyte.

In addition to reducing complementarity by reducing the base number of the invasive DNA strands in relation to the cleaved substrate fragments, other methods of reducing complementarity also may be used. For example, the invasive DNA strands may include bases that do not effectively hybridize with the bases of the cleaved substrate fragments. In another aspect, the bases from which the substrate and the invasive DNA are assembled may be selected to more weakly hybridize in relation to other base-pairs. Other methods of reducing the strength of hybridization between the cleaved substrate fragments and the invasive DNA strands as known to one of ordinary skill in the art also may be used.

### Example 5

### Confirming the Selectivity and Sensitivity of the Sensor

In a quarts UV-vis spectrophotometer cell (Hellma, Germany), 60.3 μL of 25 mM tris acetate (pH 8.2), 17 μL of 100 mM NaCl-25 mM tris acetate (pH 8.2), 1.8 μL of 0.1 mM Inva-6 invasive DNA, and 1 μL of a 0.5 mM solution containing a metal salt were combined. Samples were prepared that included the following metal salts: Pb(OAc)₂, CoCl2, ZnCl₂, CdCl₂, NiCl₂, CuCl₂, MgCl₂, and CaCl₂. After a baseline measurement, 10 μL of the aggregate mixture from Example 1 was added to each cell. This addition gave a final NaCl concentration of 30 mM, a final Inva-6 invasive DNA concentration of 2 μM for each DNA strand, and a final metal ion concentration of 5 μM for each metal tested. After complete dispersion, the extinction at 522 nm was ˜1.

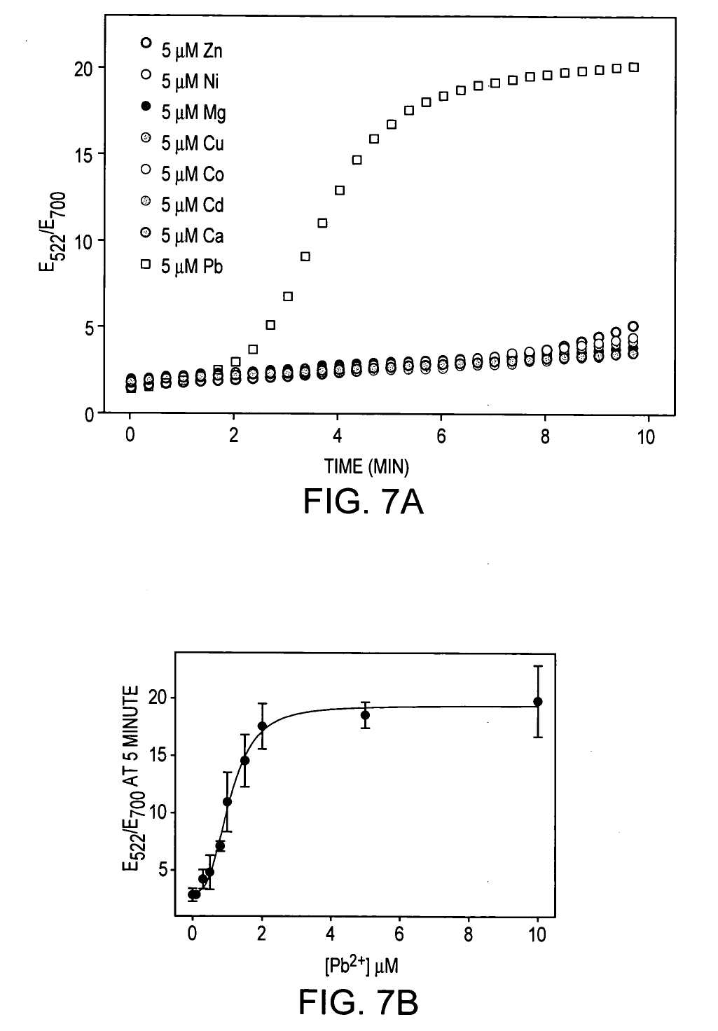

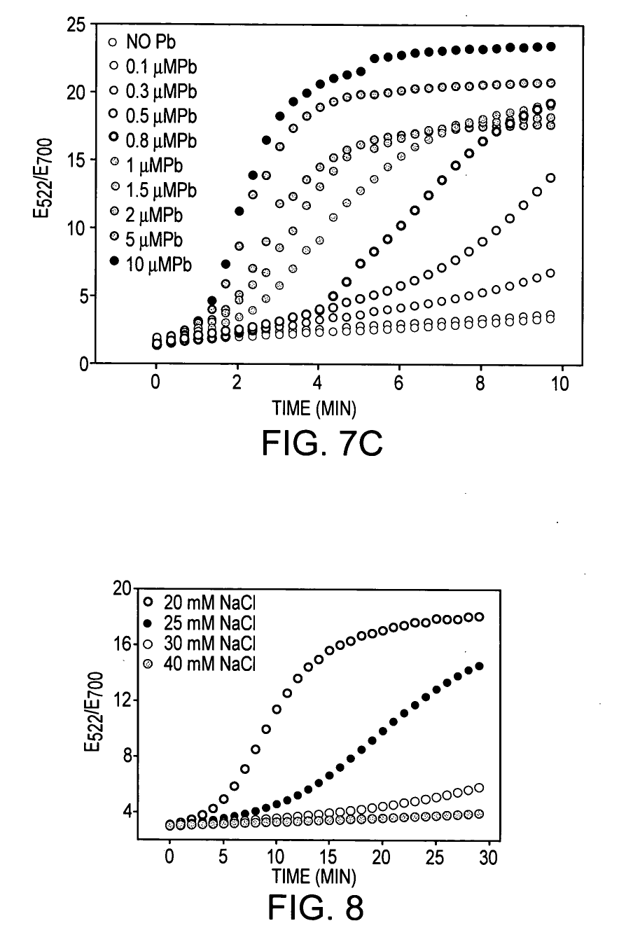

The dispersion kinetics for each metal ion was monitored as a function of time using a Hewlett-Packard 8453 spectrophotometer. FIG. 7A is a graph depicting the ratios of extinction at 522 and 700 nm plotted as a function of time. As may be seen from the plots, only Pb(II) gave significant increase in the extinction ratio as a function of time, while the other metal ions Zn(II), Co(II), Cd(II), Mg(II), Cu(II), Ni(II), and Ca(II), provided a color change consistent with the background. Therefore, the high selectivity of the sensor was confirmed.

FIG. 7B is a graph depicting the correlation between the observed extinction ratios for the color change of the sensor system and the concentration of the Pb(II) analyte after five minutes of aggregation. The exceptional linearity of the sensor system was evident from about 0.1 to about 2 μM. FIG. 7C is a graph depicting the extinction ratios for multiple Pb(II) analyte concentrations over a 10 minute time period with Inva-6. The graph demonstrates the ability of the sensor system to effectively differentiate between different analyte concentrations within a few minutes. Thus, the ability of the sensor system to provide accurate quantitative information was established.

In addition to the instrumental method of FIG. 7B, the color developed by the sensor was conveniently observed by spotting the sensor solution on an alumina TLC plate. A color progression from blue to red was observed as the concentration of the Pb(II) increased from 0 to 10 μM. The other metal ions provided a color similar to the background.

### Example 6

### Determining the Preferred Ionic Strength Environment for the Sensor

To facilitate the rapid dispersion of the aggregates from Example 1, the aggregates were suspended in a buffer containing NaCl to determine the lowest NaCl concentration capable of stabilizing the aggregates. FIG. 8 is a graph depicting the NaCl-dependent stability of the aggregates. The data were acquired on a Hewlett-Packard 8453 spectrophotometer. The buffer was 25 mM Tris acetate, pH 7.6, having NaCl concentrations of 20, 25, 30, and 40 mM. Because the sample container was a quartz UV-vis cell, instead of a 96-well plate, the extinction ratio is different from the values obtained in the prior Examples. Within half an hour, the aggregates were stable when the NaCl concentration was about 30 mM and higher. Therefore, a 30 mM NaCl solution was chosen as having an appropriate ionic strength to stabilize the aggregates while not having a substantial adverse effect on sensor response time.

### Example 7

### Aggregate Characterization

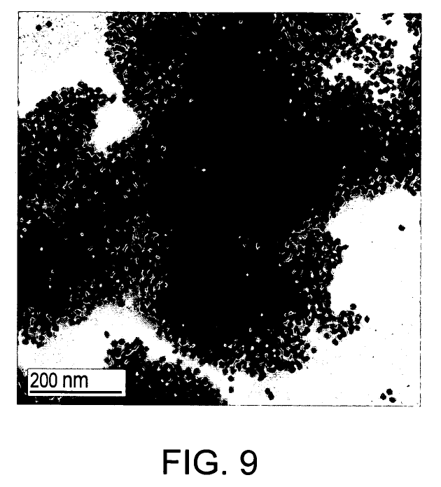

FIG. 9 is a transmission electron microscopy (TEM) image of DNAzyme-assembled 13 mm gold nanoparticle aggregates. The scale bar corresponds to 200 nm. It is clear from the image that the aggregates contain substantial numbers of gold nanoparticles.

As any person of ordinary skill in the art will recognize from the provided description, figures, and examples, that modifications and changes can be made to the preferred embodiments of the invention without departing from the scope of the invention defined by the following claims and their equivalents.

1. A sensor system for detecting an analyte, comprising:
a nucleic acid enzyme;
a substrate for the nucleic acid enzyme, comprising first polynucleotides;
first particles comprising second polynucleotides, the second polynucleotides coupled to the first particles, where the first polynucleotides are at least partially complementary to the second polynucleotides; and
invasive DNA, comprising fourth polynucleotides, the fourth polynucleotides at least partially complementary to the first polynucleotides.

2. The sensor of claim 1, further comprising second particles comprising third polynucleotides, the third polynucleotides coupled to the second particles at the 5′ terminus,
where the second polynucleotides are coupled to the first particles at the 3′ terminus and the first polynucleotides are at least partially complementary to the third polynucleotides.

3. The sensor of claim 1, where the nucleic acid enzyme comprises DNA.

4. The sensor of claim 1, where the first set of particles comprise a material selected from the group consisting of metals, semiconductors, magnetizable materials, and combinations thereof.

5. The sensor of claim 2, where the first set of particles and the second set of particles comprise gold.

6. The sensor of claim 1, the first set of particles having an average diameter from 5 nm to 70 nm.

7. The sensor of claim 1, the first set of particles having an average diameter from 10 nm to 15 nm.

8. The sensor of claim 1, where the analyte activates or deactivates the nucleic acid enzyme.

9. The sensor of claim 1, where the analyte is selected from the group consisting of Ag(I), Pb(II), Hg(II), As(III), Fe(III), Zn(II), Cd(II), Cu(II), Sr(II), Ba(II), Ni(II), Co(II), As(V), U(VI), and Cr(VI).

10. The sensor of claim 1, where the analyte comprises a metal ion having a ⁺2 formal oxidation state.

11. The sensor of claim 1, where the analyte comprises Pb(II).

12. The sensor of claim 1, where the nucleic acid enzyme comprises a polynucleotide having a sequence selected from the group consisting of SEQ ID NOS: 26-44 and conservatively modified variants thereof.

13. The sensor of claim 1, where the nucleic acid enzyme comprises a polynucleotide having a sequence of SEQ ID NO: 1 and conservatively modified variants thereof and the first polynucleotides comprise a polynucleotide having a sequence of SEQ ID NO: 3 and conservatively modified variants thereof.

14. The sensor of claim 1, where the fourth polynucleotides comprise at least two different strands, each having at least one terminal base that is complementary to at least one terminal base of a cleaved substrate strand, when the substrate is cleaved by the nucleic acid enzyme.

15. The sensor of claim 1, where the fourth polynucleotides comprise at least two different strands, each having from 2 to 10 fewer bases capable of hybridizing with a cleaved substrate strand than a fully complementary strand, when the substrate is cleaved by the nucleic acid enzyme.

16. The sensor of claim 1, where the fourth polynucleotides comprise at least two different strands, each having 6 fewer bases capable of hybridizing with a cleaved substrate strand than a fully complementary strand, when the substrate is cleaved by the nucleic acid enzyme.

17. The sensor of claim 1, where the fourth polynucleotides comprise a polynucleotide selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and conservatively modified variants thereof.

18. The sensor of claim 1, where the fourth polynucleotides comprise polynucleotides having a sequence of SEQ ID NO: 12 and conservatively modified variants thereof and SEQ ID NO: 13 and conservatively modified variants thereof.

19. A method of detecting an analyte, comprising:
combining an aggregate, a sample, and invasive DNA; and
detecting a color change responsive to the analyte, the aggregate comprising:
a substrate, comprising first polynucleotides; and
first particles comprising second polynucleotides, the second polynucleotides coupled to the first particles, where the first polynucleotides are at least partially complementary to the second polynucleotides.

20. The method of claim 19, further comprising adjusting the ionic strength of the sample.

21. The method of claim 19, where the sample and the invasive DNA are added to the aggregate.

22. The method of claim 19, where the aggregate is added to the sample and the invasive DNA.

23. The method of claim 19, where the aggregate further comprises:
second particles comprising third polynucleotides, the third polynucleotides coupled to the second particles at the 5′ terminus,
where the second polynucleotides are coupled to the first particles at the 3′ terminus and the first polynucleotides are at least partially complementary to the third polynucleotides.

24. The method of claim 23, where the particles comprise gold.

25. The method of claim 23, where the aggregate further comprises an endonuclease that comprises a binding site for the analyte, where the endonuclease is at least partially complementary to the substrate.

26. The method of claim 25, where the endonuclease comprises a nucleic acid enzyme.

27. The method of claim 26, where the nucleic acid enzyme comprises a polynucleotide having a sequence selected from the group consisting of SEQ ID NOS: 26-44 and conservatively modified variants thereof.

28. The method of claim 26, where the nucleic acid enzyme comprises a polynucleotide having a sequence of SEQ ID NO: 1 and conservatively modified variants thereof and the first polynucleotides comprise a polynucleotide having a sequence of SEQ ID NO: 3 and conservatively modified variants thereof.

29. The method of claim 26, where the invasive DNA competes with the nucleic acid enzyme to hybridize the substrate.

30. The method of claim 19, where the color change is at least 95% complete 5 minutes after combining the aggregate, the sample, and the invasive DNA.

31. The method of claim 19, where the combining occurs from 20 to 30° C.

32. The method of claim 19, where the aggregate disaggregates in response to the analyte.

33. The method of claim 32, where the response is proportional to the quantity of the analyte in the sample.

34. The method of claim 32, where the analyte activates or deactivates the nucleic acid enzyme.

35. The method of claim 19, where the analyte is selected from the group consisting of Ag(I), Pb(II), Hg(II), As(III), Fe(III), Zn(II), Cd(II), Cu(II), Sr(II), Ba(II), Ni(II), Co(II), As(V), U(VI), and Cr(VI).

36. The method of claim 19, where the analyte comprises Pb(II).

37. The method of claim 19, where the fourth polynucleotides comprise at least two different strands, each having at least one terminal base that is complementary to at least one terminal base of a cleaved substrate strand.

38. The method of claim 19, where the fourth polynucleotides comprise at least two different strands, each having from 2 to 10 fewer bases capable of hybridizing with a cleaved substrate strand than a fully complementary strand.

39. The method of claim 19, where the fourth polynucleotides comprise at least two different strands, each having 6 fewer bases capable of hybridizing with a cleaved substrate strand than a fully complementary strand.

40. The method of claim 19, where the fourth polynucleotides comprise a polynucleotide selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and conservatively modified variants thereof.

41. The method of claim 19, where the fourth polynucleotides comprise polynucleotides having a sequence of SEQ ID NO: 12 and conservatively modified variants thereof and SEQ ID NO: 13 and conservatively modified variants thereof.

42. The method of claim 19, where the sample originates from a biological source.

43. The method of claim 19, where the sample originates from an industrial waste stream.

44. The method of claim 19, where the sample originates from a water supply from which water is drawn for human consumption.

45. The method of claim 19, further comprising quantifying the color change.

46. A kit for the detection of an analyte, comprising:
a system for forming aggregates, comprising:
a substrate, comprising first polynucleotides,
first particles comprising second polynucleotides, the second polynucleotides coupled to the first particles, where the first polynucleotides are at least partially complementary to the second polynucleotides;
at least one first container containing the system for forming aggregates;
invasive DNA, comprising fourth polynucleotides, the fourth polynucleotides at least partially complementary to the first polynucleotides;
at least one second container containing the invasive DNA, where a sample may be added to a container selected from the group comprising the first container, the second container, and a third container.

47. The kit of claim 46, where the system further comprises second particles comprising third polynucleotides, the third polynucleotides coupled to the second particles at the 5′ terminus,
where the second polynucleotides are coupled to the first particles at the 3′ terminus and the first polynucleotides are at least partially complementary to the third polynucleotides.

48. The kit of claim 46, further comprising a reagent to modify the ionic strength of the sample.

49. The kit of claim 46, further comprising a reagent to modify the pH of the sample, the reagent selected from the group consisting of acids and bases.

50. The kit of claim 46, further comprising instructions to form the aggregate.

51. The kit of claim 48, further comprising instructions to modify the ionic strength of the sample.

52. The kit of claim 46, further comprising an endonuclease that comprises a binding site for the analyte, where the endonuclease is at least partially complementary to the substrate.

53. The kit of claim 52, where the endonuclease comprises a nucleic acid enzyme.

54. The kit of claim 53, where the nucleic acid enzyme comprises a polynucleotide having a sequence selected from the group consisting of SEQ ID NOS: 26-44 and conservatively modified variants thereof.

55. The kit of claim 46, where the fourth polynucleotides comprise at least two different strands, each having at least one terminal base that is complementary to at least one terminal base of a cleaved substrate strand, when the substrate is cleaved by the nucleic acid enzyme.

56. The kit of claim 46, where the fourth polynucleotides comprise at least two different strands, each having from 2 to 10 fewer bases capable of hybridizing with a cleaved substrate strand than a fully complementary strand, when the substrate is cleaved by the nucleic acid enzyme.

57. The kit of claim 46, where the fourth polynucleotides comprise at least two different strands, each having 6 fewer bases capable of hybridizing with a cleaved substrate strand than a fully complementary strand, when the substrate is cleaved by the nucleic acid enzyme.

58. The kit of claim 46, where the fourth polynucleotides comprise a polynucleotide selected from the group consisting of SEQ ID NO: 8, SEQ ID NO:9, SEQ ID NO:10, SEQ ID NO:11, SEQ ID NO: 12, SEQ ID NO: 13, SEQ ID NO: 14, SEQ ID NO: 15, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and conservatively modified variants thereof.

59. The kit of claim 46, where the fourth polynucleotides comprise polynucleotides having a sequence of SEQ ID NO: 12 and conservatively modified variants thereof and SEQ ID NO: 13 and conservatively modified variants thereof.

60. The kit of claim 46, further comprising a device to quantify a color change responsive to the disaggregation of the aggregate.

61. The kit of claim 60, where the device is selected from the group consisting of spectrophotometers and color comparators.

62. The kit of claim 60, further comprising a light-down sensor system responsive to the analyte.
